(19) United States
(12) Patent Application Publication
(10) Pub. No.: US 2022/0188753 A1
(43) Pub. Date: Jun. 16, 2022

(54) DIGITAL SUPPLY CHAIN MANAGEMENT SYSTEM

(71) Applicant: Mayo Foundation for Medical Education and Research, Rochester, MN (US)

(72) Inventors: Joseph M. Dudas, Rochester, MN (US); David Marcelleti, Rochester, MN (US); Daniel K. Schmitz, Rochester, MN (US)

(21) Appl. No.: 17/441,990

(22) PCT Filed: Mar. 26, 2020
(86) PCT No.: PCT/US2020/025033

§ 371 Date: Sep. 22, 2021

Related U.S. Application Data
(60) Provisional application No. 62/824,295, filed on Mar. 26, 2019.

(51) Int. Cl. G06Q 10/08; G16H 40/00

(52) U.S. Cl. CPC G06Q 10/087; G16H 40/00

(57) ABSTRACT

A computerized supply chain management system and method includes one or more input/output interfaces, one or more memories or databases, one or more processors communicably coupled to the one or more input/output interfaces and the one or more memories or databases, and an information management platform communicably coupled to the one or more processors. The information management platform includes a process automation component, an application programming interface management component, a dynamic analytics component, and a data management component. The information management platform manages a supply chain using one or more automated processes executed by the one or more processors.

### CROSS-REFERENCE TO RELATED APPLICATIONS

This application claims priority to and is a PCT application of U.S. Provisional Patent Application No. 62/824,295 filed on Mar. 26, 2019 and entitled “Digital Supply Chain Management System”, which is hereby incorporated by reference in its entirety.

### TECHNICAL FIELD OF THE INVENTION

The present invention relates in general to supply chain management, and more particularly, to a digital supply chain management system.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with healthcare supply chain management.

In the healthcare industry, one cost that is hidden to most is a very active and expensive supply chain. In fact, the cost of the supply chain within hospital care is second only to labor costs (about 20%-30% of the total budget). Hidden even deeper within the supply chain is the fact that more than one-third of the cost of the supply chain has nothing to do with manufacturing or developing medical products. Instead, it is the cost to sell and distribute products. On the surface, the healthcare supply chain looks much like that of any other industry. However, there is a glaring difference in the buyer and seller. In some cases, the twenty manufacturers make up more than 505 of the market; whereas, the top ten hospitals make up less than 1%-2% of that same market.

Due to the degree of fragmentation on the buyer side of the healthcare supply chain, the cost to simply sell and distribute supplies is an area of significant overhead. Not to mention an often insurmountable challenge to new entrants. Although there are many healthcare provider consolidations underway, they are nowhere close to solving the magnitude of the imbalance between the buyer and seller within the healthcare supply chain. While healthcare has adopted supply chain best practices and technology, these solutions were not designed to address these highly fragmented and inefficient markets. As a result, they do little to address the core issue.

As a result, there is a need for a digital supply chain for healthcare.

### SUMMARY OF THE INVENTION

One embodiment of the present invention provides a computerized supply chain management system that includes one or more input/output interfaces, one or more memories or databases, one or more processors communicably coupled to the one or more input/output interfaces and the one or more memories or databases, and an information management platform communicably coupled to the one or more processors. The information management platform includes a process automation component, an application programming interface management component, a dynamic analytics component, and a data management component. The information management platform manages a supply chain using one or more automated processes executed by the one or more processors.

In one aspect, one or more systems, modules, or data sources communicably coupled to the one or more input/output interfaces. In another aspect, the process automation component automates logistics and inventory management processes using robotic process automation, intelligent data capture and cognitive automation. In another aspect, the process automation component automatically identifies any purchases and inventory locations of a recalled product, generates instructions to remove the recalled product, and generates a product return for the recalled product, all in response a recall message for the recalled product. In another aspect, the process automation component automatically identifies any users or patients impacted by the recalled product. In another aspect, the application programming interface management component creates, analyzes and manages one or more application programming interfaces. In another aspect, the application programming interface management component automatically creates an item add/change/delete request using a template. In another aspect, the application programming interface management component automatically processes and provides alternatives backordered items, builds and provides requisitions, and requests and processes services. In another aspect, the dynamic analytics component provides seamless access to data within the one or more memories or databases. In another aspect, the dynamic analytics component automatically optimizes procedure-based preference card content. In another aspect, the data management component integrates, cleans and maintains data within the one or more memories or databases. In another aspect, an innovation sandbox component provides supply chain simulation and application development. In another aspect, the information management platform provides one or more functions selected from procure to pay, category management, operations, risk management, business management, and initiative management. In another aspect, the procure to pay function comprises procurement, payables, informatics and/or customer service. In another aspect, the category management function comprises performance consulting, contracting, value analysis and/or formulary. In another aspect, the operations function comprises inventory management, logistics, clinical support, operations support and/or CRM. In another aspect, the risk management function comprises financial management, business continuity, and/or audit. In another aspect, the business management function comprises business development and/or data integrity. In another aspect, the initiative management function comprises portfolio management and/or project support.

Another embodiment of the present invention provides a method for managing a supply chain that provides one or more input/output interfaces, one or more memories or databases, one or more processors communicably coupled to the one or more input/output interfaces and the one or more memories or databases, and an information management platform communicably coupled to the one or more processors, wherein the information management platform comprises a process automation component, an application programming interface management component, a dynamic analytics component, and a data management component, and manages the supply chain using one or more automated processes in the information management platform executed by the one or more processors.

In one aspect, the method further comprises one or more systems, modules, or data sources communicably coupled to the one or more input/output interfaces. In another aspect, the method further comprises automating logistics and inventory management processes using robotic process automation, intelligent data capture and cognitive automation via the process automation component. In another aspect, the method further comprises automatically identifying any purchases and inventory locations of a recalled product, generating instructions to remove the recalled product, and generating a product return for the recalled product, all in response a recall message for the recalled product via the process automation component. In another aspect, the method further comprises automatically identifying any users or patients impacted by the recalled product via the process automation component. In another aspect, the method further comprises creating, analyzing and managing one or more application programming interfaces via application programming interface management via the application programming interface management component. In another aspect, the method further comprises automatically creating an item add/change/delete request using a template via the application programming interface management component. In another aspect, the method further comprises automatically processing and providing alternatives backordered items, builds and provides requisitions, and requests and processes services via the application programming interface management component. In another aspect, the method further comprises providing seamless access to data within the one or more memories or databases via the dynamic analytics component. In another aspect, the method further comprises automatically optimizing procedure-based preference card content via the dynamic analytics component. In another aspect, the method further comprises integrating, cleaning and maintaining data within the one or more memories or databases via the data management component. In another aspect, the method further comprises providing supply chain simulation and application development via an innovation sandbox component. In another aspect, the method further comprises providing one or more functions selected from procure to pay, category management, operations, risk management, business management, and initiative management via the information management platform. In another aspect, wherein the procure to pay function comprises procurement, payables, informatics and/or customer service. In another aspect, wherein the category management function comprises performance consulting, contracting, value analysis and/or formulary. In another aspect, wherein the operations function comprises inventory management, logistics, clinical support, operations support and/or CRM. In another aspect, wherein the risk management function comprises financial management, business continuity, and/or audit. In another aspect, wherein the business management function comprises business development and/or data integrity. In another aspect, wherein the initiative management function comprises portfolio management and/or project support.

### BRIEF DESCRIPTION OF THE DRAWINGS

For a more complete understanding of the features and advantages of the present invention, reference is now made to the detailed description of the invention along with the accompanying figures and in which:

FIG. 1 is a block diagram of a Digital Supply Chain (DSC) system in accordance with one embodiment of the present invention;

FIG. 2 is a block diagram of a DSC system in accordance with one embodiment of the present invention;

FIG. 3 is a block diagram of a development process for the DSC in accordance with one embodiment of the present invention;

FIG. 4 illustrates a supply chain data management example in accordance with one embodiment of the present invention;

FIG. 5 illustrates a supply chain process automation example in accordance with one embodiment of the present invention;

FIG. 6 illustrates a supply chain data analytics example in accordance with one embodiment of the present invention;

FIG. 7 illustrates a supply chain API example in accordance with one embodiment of the present invention;

FIG. 8 illustrates a supply chain innovation sandbox example in accordance with one embodiment of the present invention;

FIG. 9 is a block diagram of a computerized supply chain management system in accordance with another embodiment of the present invention; and

FIG. 10 is a block diagram of a method for managing a supply chain in accordance with another embodiment of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

A traditional business (and today's healthcare supply chain) works like a “Pipeline”. Value is produced upstream and consumed downstream, creating a sequential flow of value, much like water flowing through a pipe. This type of system is very efficient when dealing with large batches (e.g., Walmart, Proctor & Gamble, etc.). “Platforms” however work vastly different, which is why “Digital” is so disruptive. Platforms allow participants to exchange value without being bound to a specific sequence. The infrastructure is open and artificial intelligence is leveraged to match supply and demand. This system is very efficient when dealing with real-time transactions (e.g., Uber, Airbnb, the modern day traveler, etc.). In a nutshell, digital platforms are designed to work well in fragmented and real-time transactional markets.

The current healthcare supply chain system is a pipeline that is centered on enterprise resource planning (ERP) and electronic data interchange (EDI). There are hundreds of traditional interfaces all centered on a highly automated yet very sequential batch process. In contrast, a digital supply chain (DSC) in accordance with the present invention is a “platform” as opposed to a “pipe”. Functions are optimized through the use of a common information management platform (IMP). In some embodiments, the DSC provides value by reducing physical labor, reducing transactional effort, and accelerating contract savings.

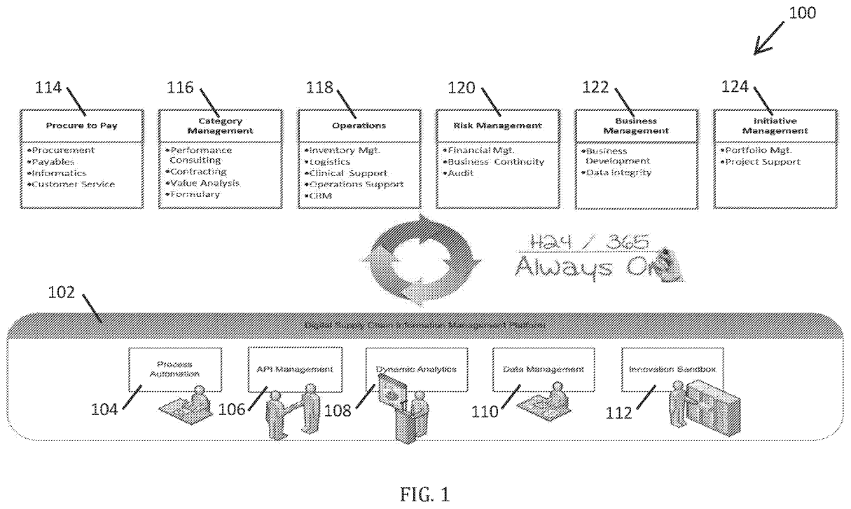

Now referring to FIG. 1, a block diagram of a Digital Supply Chain (DSC) system 100 in accordance with one embodiment of the present invention is shown. The DSC system 100 includes an integrated DSC information management platform (IMP) 102 that seamlessly connects and coordinates each process so that the entire system 100 can be optimized. The DSC system 100 ingests, enriches, harmonizes and acts on the vast amount of information that largely goes unused today. In addition, the DSC system 100 uses highly automated and concurrent smart processes instead of single threaded applications. Furthermore, it enables the delivery of the products, information and services needed to manage health delivery across the entire ecosystem at a speed, reach and scale that is characterized by a fully mature shared service.

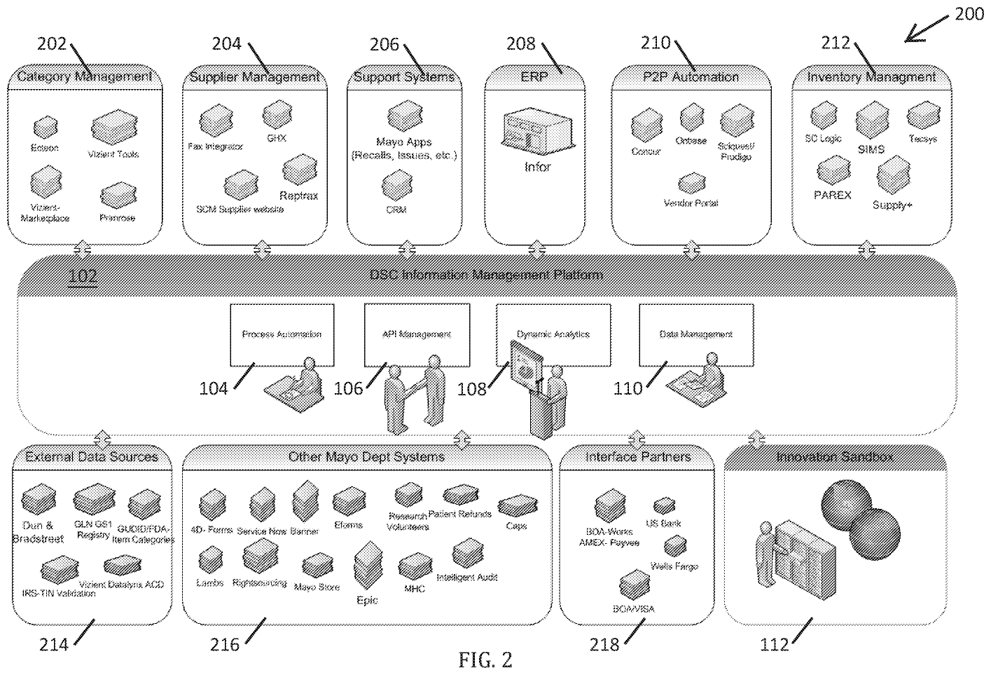

Functions are optimized through the use of a common DSC IMP 102, which is made up of four main features that are capable of working together in real-time. They are process automation 104 (e.g., RPA/BPM, etc.), API management/process integration 106 (e.g., Block Chain, etc.), dynamic analytics 108 (e.g., IOT, Big Data, ML, etc.), and data management 110 (PIM, KMS). An innovation sandbox 112 can be part of the DSC IMP 102 or a separate process as shown in FIG. 2. This type of system will provide new levels of productivity, scale and value.

The DSC IMP 102 continuously provides various functions, which can include procure to pay 114, category management 116, operations 118, risk management 120, business management 122, and/or initiative management 124. Note that the DSC IMP 102 can provide other functions. Procure to pay 114 can include procurement, payables, informatics and/or customer service. Category management 116 can include performance consulting, contracting, value analysis and/or formulary. Operations 118 can include inventory management, logistics, clinical support, operations support and/or CRM. Risk management 120 can include financial management, business continuity, and/or audit. Business management 122 may include business development and/or data integrity. Initiative management 124 may include portfolio management and/or project support.

Platforms, like the DSC, can optimize “interactions” across other business functions. For example, make-to-order (MTO) is a manufacturing process in which manufacturing starts only after receipt of a customer's order. MTO can be used to manage high-end and very expensive medical devices in the DSC. A stockless or reduced stock supply chain can significantly reduce the cost of sales and distribution of medical products. In another example, virtual care is a method, or methods, by which physicians and other health care providers communicate with patients without physically seeing them in the office. A virtual visit could conclude with a virtual care/supply chain “Digital” integration where supplies are sent directly to the patient's home. This type of integration and service could change the perception of patients, especially if is available 24/7. In yet another example, precision medicine is a medical model that proposes the customization of healthcare, with medical decisions, treatments, practices, or products being tailored to the individual patient. A practice/supply chain/research and development “Digital” integration could accelerate the development of products that are manufactured and administered for individuals (e.g., based on their DNA, etc.). A “Precision Supply Chain” could support “Precision Medicine”.

Platforms provide an open infrastructure in which participants can co-create and exchange value with each other unbound by a specific sequence. Participants (within each function) work together, in a relatively unencumbered manner and encouraged to meet the needs of the customer. Each participant either with a shared platform or able to “openly” to connect as needed. For example, smart apps provide a formless supply chain that takes the robot out of the human. In another example, comparison agents capture and share product expertise. The open infrastructure provides both the tools and support to effectively bring producers and consumers together (enablement as opposed to control) to ideally match supply with demand. APIs act as self-service contracts and data-based algorithms allocate resources as well as make decisions.

The DSC IMP has Many Benefits, Such as:

Services multiple roles. Clinicians, category managers, buyers, operators, sellers, distributors.

Encourages use (users and frequency). Enables product, contract, purchase, inventory and utilization interactions.

Strive for scale but ensures context and quality. Deliver demand management data, tools & features that provide exceptional context.

Enables processes that deliver value. Efficiently manages demand, reduces waste, delivers return on investment and rates stakeholders. Platform value could be captured through a subscription as well as data deposit.

The DSC creates an ecosystem, infrastructure (tools, services and rules) and data that is used to effectively and efficiently manage supply and demand. It essentially obsoletes VANs (value added networks aka EDI exchanges). Through APIs, the DSC serves as an interface between stakeholder applications and helps them interact effectively, similarly to how a user interface helps humans interact with computers (real-time interactions). Because interactions are real-time, a series of value added steps (through Process Automation, no-code development) can be executed at the time of the transaction. A good example of this is in retail online purchasing. When you enter your credit card information to buy an item online, the web store uses an API to send that information to a remote application, which then verifies it to ensure that it's correct. After confirming that your credit card info is valid, the application sends back a confirmation that the order can be processed (in seconds). In the DSC transactions are conversational and therefore could create the types of Commercial SCM interactions that we experience as online consumers. Coupled with real-time access to Big Data and AI, transactions can be made not only conversational but also intelligent. Additionally, electronic control towers can be established that monitor activity and can help quickly respond to issues and make accurate projections about numerous parts of the supply chain.

Now referring to FIG. 2, a block diagram of a DSC system 200 in accordance with one embodiment of the present invention is shown. The DSC system 200 is a digital IMP 102 made up of four core components (Process Automation 104, API Management 106, Dynamic Analytics 108 and Data Management 110) communicably coupled to other systems, components and modules, data sources, partners and vendors, etc. Coupled with a modular design principle and a deliberate attention to innovation through an innovation sandbox 112, this architecture provides an almost unlimited technical infrastructure that will have a dramatic ripple effect on healthcare business model(s) and human capital. Following are more detailed descriptions of each of the components:

Process Automation 104: There are three categories of process automation 104: Robotic Process Automation (RPA), Intelligent Data Capture (IDC) and Cognitive Automation. RPA for the most part automates clerical work to handle high-volume, repeatable tasks that previously required humans to perform. It operates by mapping a process in the tool for the software “robot” to follow computer pathways between screens and various data repositories. IDC systems learn to recognize, classify, extract key index information, and automatically route it to the appropriate workflows. However, the real difference to what we do today is combining automation, orchestration, advanced analytics into what is called Cognitive Automation. Cognitive Automation leverages technologies such as Machine Learning, Natural Language Processing and Artificial Intelligence. Together these technologies allow SCM to “automate anything” and can be extended from “digital automation” to “physical automation” such as logistics and inventory management processes.

API Management 106: A modular system allows for flexibility in how functionality is accessed and leveraged. Key to a modular system is how they are connected which is through an API. An API is an application programming interface, or an interface that enables controlled interaction between the different software programs that are being used. Generally speaking these interfaces allow for real-time (or just-in-time) access to application functionality. The better the API, the more the system can be integrated to run the business more efficiently. API Management 106 enables the creation, analysis, and management of APIs in a secure and scalable environment.

Dynamic Analytics 108: Dynamic Analytics 108 (also referred to as “Modern Analytics”) allows seamless access to just-in-time data across the landscape of technologies that support all business processes. Analytics will adapt to the needs of employees and customers instead of forcing them to adopt traditional approaches to producing and consuming data. Data and analytics must allow for fewer constraints in how analysis is done, and more choices in how it can be used. Today (for SCM), analytics are constrained by an old model that was based on the computers of the past. In the future, we will seek to leverage the latest advances to deploy intelligence, data science and action without the need for a physical data warehouse.

Data Management 110: Effective data management enables you to integrate, clean and maintain your data. To obtain the clean, complete and current data required for accurate reporting and analytics or other operational use cases, you must have a comprehensive data management foundation. Data management 110 includes understanding, cleansing, integrating, governing, mastering and monitoring data as a strategic asset. Functions that support this area include the following:

Data Access—Access and distribute data anywhere (structured and unstructured, at rest and streaming).

Data Knowledge—Capture a complete data picture that allows combining and interpreting data from different sources.

Data Preparation—Provide self-service access to business users to prepare, blend and cleanse data without burdening a central function.

Data Quality—Ensure data is accurate, consistent and trusted (fits the purpose).

Data Governance—Oversight and authority to ensure that strategic data is formally managed.

Master Data Management—Manage a single view of important data that is used by all areas of the supply chain.

Innovation Sandbox 112: A sandbox is a place where you can play without consequences, and where you can destroy and rebuild your successive imperfect attempts at creating something. So it may be any sort of environment (a lab, a piece of software, a separate computer, etc.) where you experiment. In the context of the DSC 200, the IMP 102 self-generates this place, which allows for supply chain simulation as well as application development.

The other systems, components and modules, data sources, partners and vendors may include, but are not limited to:

Category Management 202 (e.g., Ecleon, Vizient Tools, Vizient Marketplace, Primrose, etc); Supplier Management 204 (e.g., Fax Integrator, GHX, SCM Supplier website, Reptrax, etc.);

Support Systems 206 (e.g., Mayo Apps (Recalls, Issues, etc.), CRM, etc.);

ERP 208 (e.g., Infor, etc.);

P2P Automation 210 (e.g., Concur, Onbase, Sciquest/Prodigo, Vendor Portal, etc.);

Inventory Management 212 (e.g., SC Logic, SIMS, Tecsys, PAREX, Supply+, etc.);

External Data Sources 214 (e.g., Dun & Bradstreet, GLN GS1 Registry, GUIDID/FDA Item Categories, IRS-TIN Validation, Vizient Datalynx ACD, etc.);

Other Mayo Dept Systems 216 (e.g., 4D-Forms, Service Now, Banner, Eforms, Research Volunteers, Patient Refunds, Caps, Lambs, Rightsourcing, Mayo Store, Epic, MHC, Intelligent Audit, etc.); and/or

Interface Partners 218 (e.g., BOA-Works, AMEX-Payvee, US Bank, Wells Fargo, BOA/VISA, etc.).

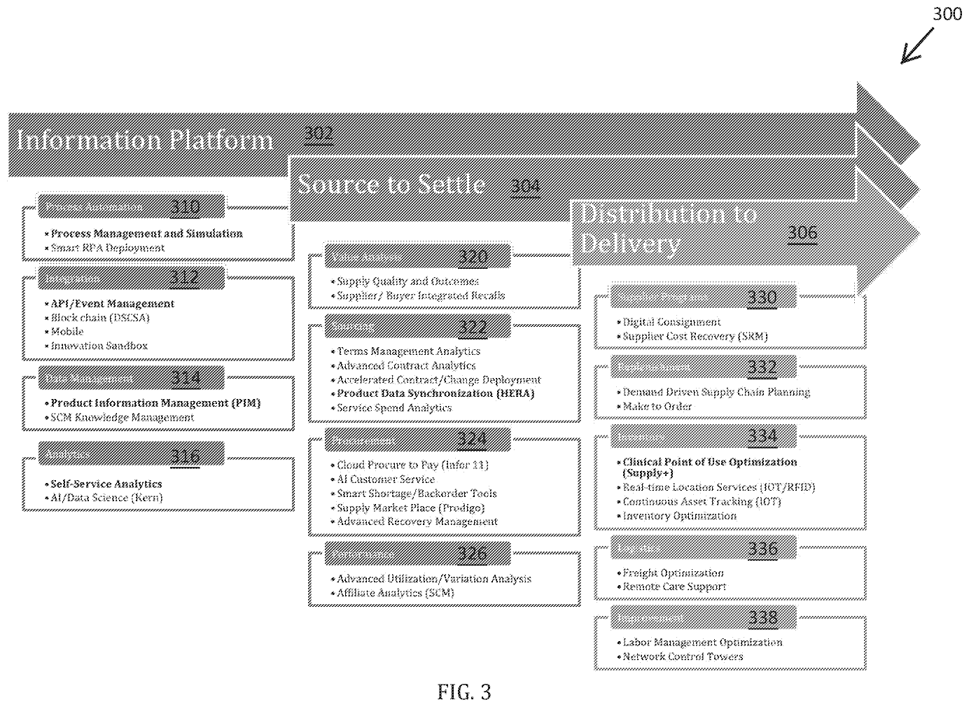

Referring now to FIG. 3, a block diagram of a development process 300 for the DSC in accordance with one embodiment of the present invention is shown. The development process 300 includes three activities: (1) Information Platform 302; (2) Source to Settle 304; and (3) Distribution to Delivery 306. The Information Platform 302 includes:

Process Automation 310 (e.g., Process Management and Simulation, Smart RPA Deployment, etc.);

Integration 312 (e.g., API/Event Management, Block chain (DSCSA), Mobile, Innovation Sandbox, etc.);

Data Management 314 (e.g., Product Information Management (PIM), SCM Knowledge Management, etc.); and

Analytics 316 (e.g., Self-Service Analytics, AI/Data Science (Kern), etc.).

The Source to Settle 304 includes:

Value Analysis 320 (e.g., Supply Quality and Outcomes, Supplier/Buyer Integrated Recalls, etc.);

Sourcing 322 (e.g., Terms Management Analytics, Advanced Contract Analytics, Accelerated Contract/Change Deployment, Product Data Synchronization (HERA), Service Spend Analytics, etc.);

Procurement 324 (e.g., Cloud Procure to Pay (Infor 11), AI Customer Service, Smart Shortage/Backorder Tools, Supply Market Place (Prodigo), Advanced Recovery Management, etc.); and

Performance 326 (e.g., Advanced Utilization/Variation Analysis, Affiliate Analytics (SCM), etc.).

The Distribution to Delivery 306 includes:

Supplier Programs 330 (e.g., Digital Consignment, Supplier Cost Recovery (SRM), etc.);

Replenishment 332 (e.g., Demand Driven Supply Chain Planning, Make to Order, etc.);

Inventory 334 (e.g., Clinical Point of Use Optimization (Supply+), Real-time Location Services (IOT/RFID), Continuous Asset Tracking (IOT), Inventory Optimization, etc.);

Logistics 336 (e.g., Freight Optimization, Remote Care Support, etc.); and

Improvement 338 (e.g., Labor Management Optimization, Network Control Towers, etc.).

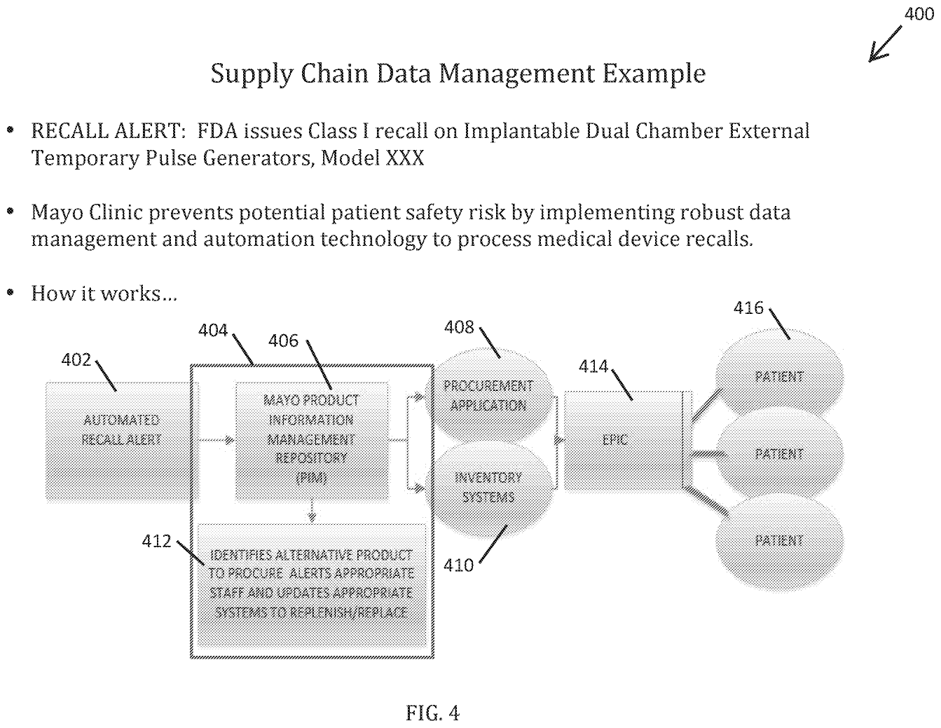

Now referring FIG. 4, a supply chain data management example 400 in accordance with one embodiment of the present invention is shown. In this example, Mayo receives an application programming interface (API) call 402 from the Food & Drug Administration (FDA) alerting of a Class I recall. Robotic Process Automation (RPA) 404 searches the May Product Information Management Repository (PIM) 406 by utilizing product attributes to identify impacted products and kick off an automated event to identify the purchases via procurement application 408 and the location of impacted devices via inventory systems 410. When inventory is located, an alert appears on inventory management dashboards with instructions to remove the product, and uses an automated ‘event manager’ to kick off a product return in block 412. The BOT also identifies the appropriate substitution utilizing cross-reference attributes in the PIM repository, and alerts appropriate SCM staff with instructions on replacing the recalled products in block 412. The EHR application 414 was alerted with the recalled serial numbers tied to impacted patient cases 416, and kicks off appropriate automated notifications to the applicable procedural areas, and patient, if applicable. A value analysis team could monitor the whole process electronically and be available online if and when needed. By collecting accurate product data, and utilizing automation and API tools, the recall process is fast, accurate and efficient. The PIM repository 406 stores information gathered during the contracting and value analysis processes along with data provided by manufacturers, third parties and consumers.

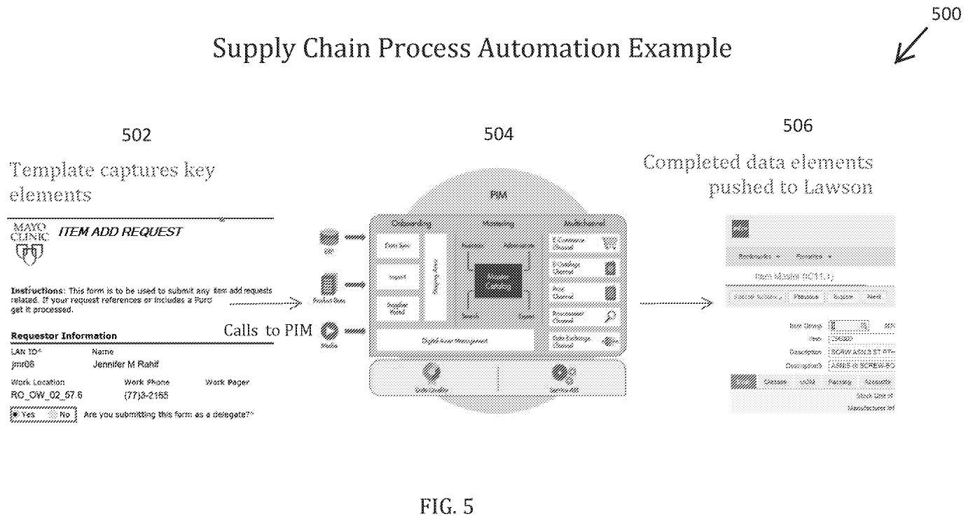

Referring now to FIG. 5, a supply chain process automation example 500 in accordance with one embodiment of the present invention is shown. This example involves an Item Add/Change/Delete request process. A smart form 502 is created and serves as a template to gather the necessary data elements form the item add from the requestor. A business process automation agent (BPA) or robot monitors key databases and events and uses the form to automatically start the process. The BPA tool triggers a series of automated processes (business rules coupled with sophisticated analytics) to gather are used to gather relevant/required data elements for the item via public and Mayo proprietary data sources 504. Manual routing is eliminated. Augmented human intelligence is used to enhance data elements. The completed data elements are pushed to Lawson 506 and an auto-notification is sent to the requestor.

Supply chain process automation provides numerous advantages, such as higher production rates, increased productivity, more efficient use of materials, better product quality, improved safety, and reduced lead times. Key benefits include the following: automate manual tasks in a process; eliminate keying errors; speed up processes; link applications together; systematically enforce ICE controls; and ensure data integrity

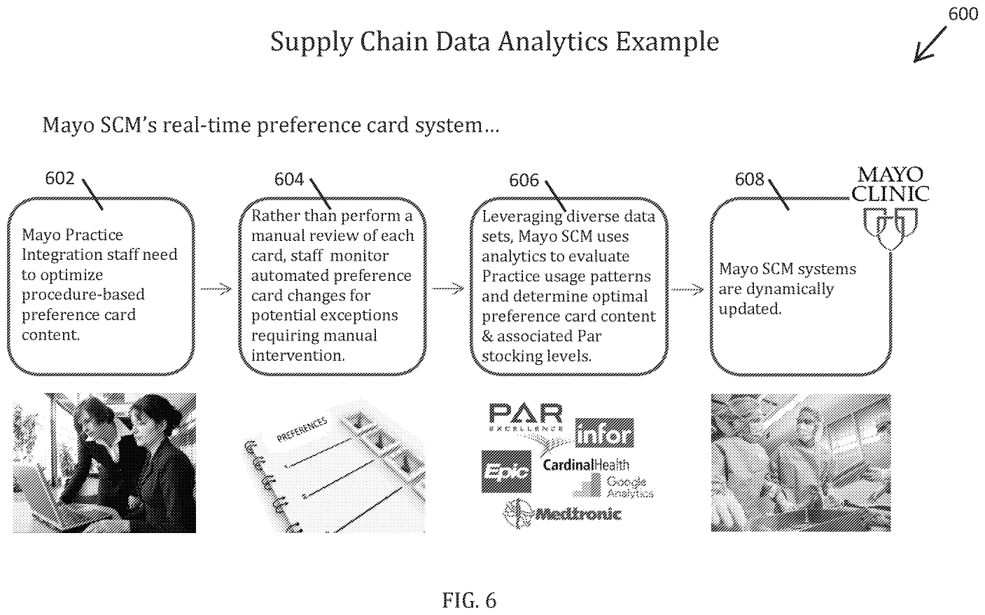

Now referring to FIG. 6, a supply chain data analytics example 600 in accordance with one embodiment of the present invention is shown. Mayo Practice Integration staff need to optimize procedure-based preference card content in block 602. Rather than perform a manual review of each card, the staff monitor automated preference card changes for potential exceptions requiring manual intervention in block 604. Leveraging diverse data sets, Mayo SCM uses analytics, such as machine learning, to evaluate practice usage patterns and determine optimal preference card content and associated Par stocking levels in block 606. The May SCM systems are dynamically updated in block 608.

Systematic evaluation of multiple patient care variables enables Mayo to utilize both leading and lagging indicators to more accurately predict demand and dynamically set supply management levels. This approach helps to: maximize product availability at the point of care; improve inventory management and labor efficiency; and drive automated, data-driven processes. Note that manual involvement of Mayo staff in this process is limited to exception management.

Dynamic Analytics has multiple benefits. First and foremost, it supports the notion of end-to-end processes that are fully automated and intelligent (the processes know how to handle exceptions). Analytics move in lockstep with business change and growth as the model can be reconfigured without a lengthy and costly data warehousing project. Modern analytics are not something that runs after the fact but rather, proactively identifies opportunities and vulnerabilities as they are about to happen in the supply chain. Key benefits can be summarized as the following: flexibility and agility; trusted and accurate (governed); architected for growth, change and continuous improvement; and eliminates the need to remodel data structures.

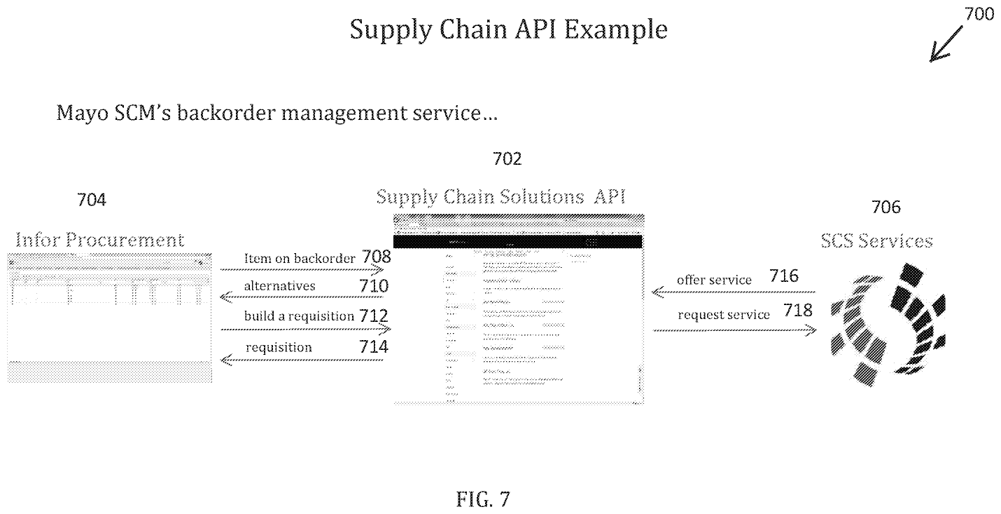

Referring now to FIG. 7, a supply chain API example 700 in accordance with one embodiment of the present invention is shown. The Supply Chain Solutions API 702 is integrated with Infor procurement service 704 and SCS Services 706. The Supply Chain Solutions API 702 interacts with Infor procurement service 704 for items on backorder 708, alternatives 710, build a requisition 712 and requisition 714. The Supply Chain Solutions API 702 interacts with SCS Services 706 to offer service 716 and request service 718. Moreover, the Supply Chain Solutions API 702 lets users order an alternative item from the procurement service without having to switch to the Supply Chain Solutions' app. Mayo applied modular design principles by isolating and their SCS Backorder Management Service. Supply Chain Solutions API 702 is a single and standardized API to access that service and allow anyone, anywhere to make a request and service it “real-time”. The service will also suggest alternative products as well as create a requisition.

API Management provides numerous benefits:

Redesign and reassemble functionality: Companies today can take advantage of the public and private cloud to weave new applications and services quickly and seamlessly.

Enable interaction (internally and externally) with core digital processes: API Management allows for hybrid technology, process and R&D. New innovative and very effective functionality can be deployed and shared that was never before possible.

Resilience to change: Modular design isolates critical functions so that change outside of the function is minimized the inputs and outputs.

Scalability: By sharing services digitally across a broad market cost per transaction is significantly curved. Those delivering services are incented to invest and those consuming services are able to take advantage of services that only large organizations could afford.

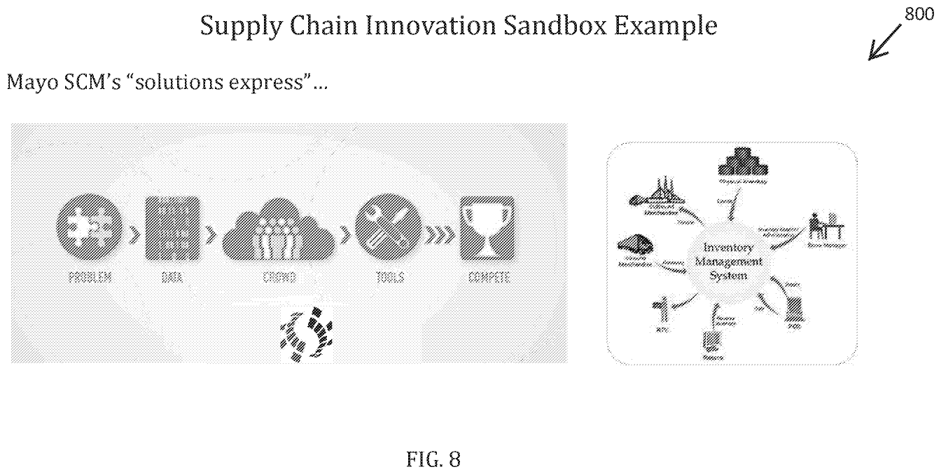

Now referring to FIG. 8, a supply chain innovation sandbox example 800 in accordance with one embodiment of the present invention is shown. The Mayo DSC launches an Innovation Sandbox as a platform for solving difficult problems. The initial launch posts an inventory management optimization problem and a complete data set representing current state. A team from the Kern Center submits a solution that improves on the current EOQ method. Innovation is critical to the Digital Supply Chain. In today's environment staff and leadership often give up on great ideas. The innovation sandbox is specifically designed to share problems, data and tools. Today innovation is not easy. Navigating project approvals and data access rights can stifle creativity. In fact, most staff give and continue with status quo as opposed to inventing. The Innovation Sandbox makes innovation easy as it encourages engagement as opposed to controlling it. The overall intellectual capability today is limited by both intended and unintended constraints. The Innovation Sandbox removes barriers and creates a new culture of engagement.

Innovation Sandboxes enable bottom-up innovation, that is, the ability for concepts and solutions to form at any level in the organization. This provides senior decision makers with a holistic survey of innovative capabilities and knowledge across the company.

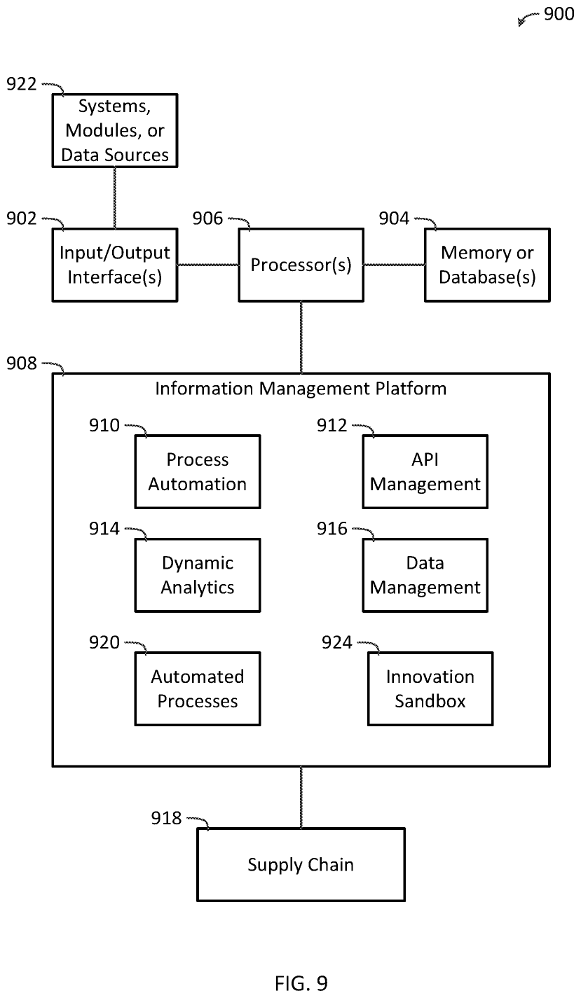

Referring now to FIG. 9, a block diagram of a computerized supply chain management system 900 in accordance with one embodiment of the present invention is shown. The computerized supply chain management system 900 includes one or more input/output interfaces 902, one or more memories or databases 904, one or more processors 906 communicably coupled to the one or more input/output interfaces 902 and the one or more memories or databases 904, and an information management platform 908 communicably coupled to the one or more processors 906. The information management platform 908 includes a process automation component 910, an application programming interface (API) management component 912, a dynamic analytics component 914, and a data management component 916. The information management platform 908 manages a supply chain 918 using one or more automated processes 920 executed by the one or more processors 906.

In one aspect, one or more systems, modules, or data sources 922 are communicably coupled to the one or more input/output interfaces 902. In another aspect, the process automation component 910 automates logistics and inventory management processes using robotic process automation, intelligent data capture and cognitive automation. In another aspect, the process automation component 910 automatically identifies any purchases and inventory locations of a recalled product, generates instructions to remove the recalled product, and generates a product return for the recalled product, all in response a recall message for the recalled product. In another aspect, the process automation component 910 automatically identifies any users or patients impacted by the recalled product. In another aspect, the application programming interface management component 912 creates, analyzes and manages one or more application programming interfaces. In another aspect, the application programming interface management component 912 automatically creates an item add/change/delete request using a template. In another aspect, the application programming interface management component 912 automatically processes and provides alternatives backordered items, builds and provides requisitions, and requests and processes services. In another aspect, the dynamic analytics component 914 provides seamless access to data within the one or more memories or databases. In another aspect, the dynamic analytics component 914 automatically optimizes procedure-based preference card content. In another aspect, the data management component 916 integrates, cleans and maintains data within the one or more memories or databases. In another aspect, an innovation sandbox component 924 provides supply chain simulation and application development. In another aspect, the information management platform 908 provides one or more functions selected from procure to pay, category management, operations, risk management, business management, and initiative management. In another aspect, the procure to pay function comprises procurement, payables, informatics and/or customer service. In another aspect, the category management function comprises performance consulting, contracting, value analysis and/or formulary. In another aspect, the operations function comprises inventory management, logistics, clinical support, operations support and/or CRM. In another aspect, the risk management function comprises financial management, business continuity, and/or audit. In another aspect, the business management function comprises business development and/or data integrity. In another aspect, the initiative management function comprises portfolio management and/or project support.

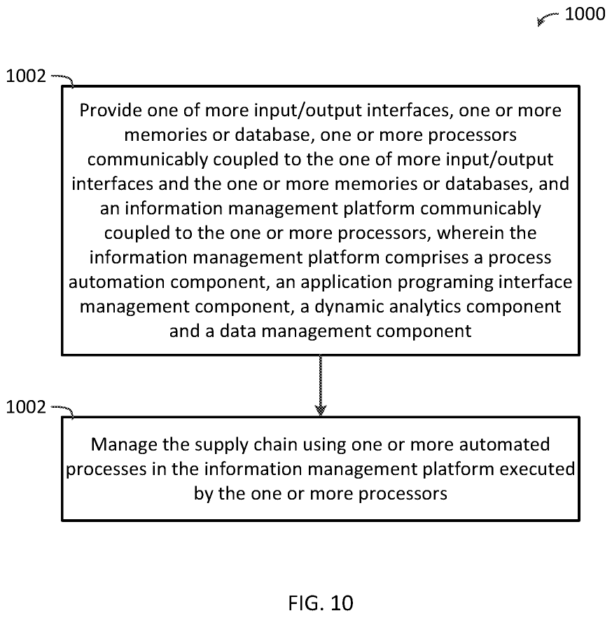

Now referring to FIG. 10, a block diagram of a method 1000 for managing a supply chain in accordance with another embodiment of the present invention is shown. One or more input/output interfaces, one or more memories or databases, one or more processors communicably coupled to the one or more input/output interfaces and the one or more memories or databases, and an information management platform communicably coupled to the one or more processors are provided in block 1002. The information management platform comprises a process automation component, an application programming interface management component, a dynamic analytics component, and a data management component. The supply chain is managed in block 1004 using one or more automated processes in the information management platform executed by the one or more processors.

In one aspect, the method further comprises one or more systems, modules, or data sources communicably coupled to the one or more input/output interfaces. In another aspect, the method further comprises automating logistics and inventory management processes using robotic process automation, intelligent data capture and cognitive automation via the process automation component. In another aspect, the method further comprises automatically identifying any purchases and inventory locations of a recalled product, generating instructions to remove the recalled product, and generating a product return for the recalled product, all in response a recall message for the recalled product via the process automation component. In another aspect, the method further comprises automatically identifying any users or patients impacted by the recalled product via the process automation component. In another aspect, the method further comprises creating, analyzing and managing one or more application programming interfaces via application programming interface management via the application programming interface management component. In another aspect, the method further comprises automatically creating an item add/change/delete request using a template via the application programming interface management component. In another aspect, the method further comprises automatically processing and providing alternatives backordered items, builds and provides requisitions, and requests and processes services via the application programming interface management component. In another aspect, the method further comprises providing seamless access to data within the one or more memories or databases via the dynamic analytics component. In another aspect, the method further comprises automatically optimizing procedure-based preference card content via the dynamic analytics component. In another aspect, the method further comprises integrating, cleaning and maintaining data within the one or more memories or databases via the data management component. In another aspect, the method further comprises providing supply chain simulation and application development via an innovation sandbox component. In another aspect, the method further comprises providing one or more functions selected from procure to pay, category management, operations, risk management, business management, and initiative management via the information management platform. In another aspect, wherein the procure to pay function comprises procurement, payables, informatics and/or customer service. In another aspect, wherein the category management function comprises performance consulting, contracting, value analysis and/or formulary. In another aspect, wherein the operations function comprises inventory management, logistics, clinical support, operations support and/or CRM. In another aspect, wherein the risk management function comprises financial management, business continuity, and/or audit. In another aspect, wherein the business management function comprises business development and/or data integrity. In another aspect, wherein the initiative management function comprises portfolio management and/or project support.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A computerized supply chain management system comprising:
one or more input/output interfaces;
one or more memories or databases;
one or more processors communicably coupled to the one or more input/output interfaces and the one or more memories or databases;
an information management platform communicably coupled to the one or more processors, wherein the information management platform comprises a process automation component, an application programming interface management component, a dynamic analytics component, and a data management component; and
the information management platform manages a supply chain using one or more automated processes executed by the one or more processors.

2. The system of claim 1, further comprising one or more systems, modules, or data sources communicably coupled to the one or more input/output interfaces.

3. The system of claim 1, wherein the process automation component automates logistics and inventory management processes using robotic process automation, intelligent data capture and cognitive automation.

4. The system of claim 1, wherein the process automation component automatically identifies any purchases and inventory locations of a recalled product, generates instructions to remove the recalled product, and generates a product return for the recalled product, all in response a recall message for the recalled product.

5. The system of claim 1, wherein the process automation component automatically identifies any users or patients impacted by the recalled product.

6. The system of claim 1, wherein the application programming interface management component creates, analyzes and manages one or more application programming interfaces.

7. The system of claim 1, wherein the application programming interface management component automatically creates an item add/change/delete request using a template.

8. The system of claim 1, wherein the application programming interface management component automatically processes and provides alternatives backordered items, builds and provides requisitions, and requests and processes services.

9. The system of claim 1, wherein the dynamic analytics component provides seamless access to data within the one or more memories or databases.

10. The system of claim 1, wherein the dynamic analytics component automatically optimizes procedure-based preference card content.

11. The system of claim 1, wherein the data management component integrates, cleans and maintains data within the one or more memories or databases.

12. The system of claim 1, further comprising an innovation sandbox component provides supply chain simulation and application development.

13. The system of claim 1, wherein the information management platform provides one or more functions selected from procure to pay, category management, operations, risk management, business management, and initiative management.

14. The system of claim 13, wherein the procure to pay function comprises procurement, payables, informatics and/or customer service.

15. The system of claim 13, wherein the category management function comprises performance consulting, contracting, value analysis and/or formulary.

16. The system of claim 13, wherein the operations function comprises inventory management, logistics, clinical support, operations support and/or CRM.

17. The system of claim 13, wherein the risk management function comprises financial management, business continuity, and/or audit.

18. The system of claim 13, wherein the business management function comprises business development and/or data integrity.

19. The system of claim 13, wherein the initiative management function comprises portfolio management and/or project support.

20. A method for managing a supply chain comprising:
providing one or more input/output interfaces, one or more memories or databases, one or more processors communicably coupled to the one or more input/output interfaces and the one or more memories or databases, and an information management platform communicably coupled to the one or more processors, wherein the information management platform comprises a process automation component, an application programming interface management component, a dynamic analytics component, and a data management component; and
managing the supply chain using one or more automated processes in the information management platform executed by the one or more processors.

21-38. (canceled)
